(19) United States
(12) Patent Application Publication
(10) Pub. No.: US 2023/0316407 A1
(43) Pub. Date: Oct. 5, 2023

(54) SYSTEM, METHOD, AND COMPUTER PROGRAM FOR STREAMLINING AUTHORIZATION FOR MEDICAL PAYMENT ADJUDICATION

(71) Applicant: Legrande Corporation, Rexburg, ID (US)

(72) Inventors: Anthony Sine, Laguna Beach, CA (US); Daniel Kevin Richards, Sandy, UT (US)

(21) Appl. No.: 17/709,209

(22) Filed: Mar. 30, 2022

(51) Int. Cl. G06Q 40/08; G16H 50/20; G16H 20/10

(52) U.S. Cl. CPC G06Q 40/08; G16H 20/10; G16H 50/20

(57) ABSTRACT

A system, method, and non-transitory computer readable storage medium are provided for streamlining authorization for medical payment adjudication. In use, a treatment fulfillment request associated with a patient, and data associated with a medical model containing a ranking of at least two prescriptions associated with the treatment fulfillment request are received from a medical provider. Next, an adjudication status of at least one prescription of the at least two prescriptions is determined. When the adjudication status of one prescription of the at least two prescriptions indicates that the one prescription is allowed by the medical insurance provider, submit the one prescription for fulfillment to a pharmacy provider as a clean claim submission. When the adjudication status of each prescription of the at least two prescriptions indicates that each prescription of the at least two prescriptions is not allowed by the medical insurance provider, request additional data for the medical model from the medical provider.

### RELATED APPLICATIONS

The present application incorporates by reference the following applications in their entirety for all purposes: U.S. application Ser. No. 17/510,166 entitled “SYSTEM, METHOD, AND COMPUTER PROGRAM FOR RECOMMENDED MEDICAL TREATMENTS BASED ON DATA MINING” filed Oct. 25, 2021 (docket number LEG1P001), and U.S. application Ser. No. 17/510,192 entitled “SYSTEM, METHOD, AND COMPUTER PROGRAM FOR ASYNCHRONOUS AND SYNCHRONOUS MEDICAL INTERACTIONS BASED ON DATA MINING” filed Oct. 25, 2021 (docket number LEG1P002).

### FIELD OF THE INVENTION

The present invention relates to automated workflows, and more particularly to automated workflows for medical payment adjudication.

### BACKGROUND

Currently, adjudication of medical benefit claims can be laborious and time intensive. For example, a medical provider may write a prescription for a patient. The patient takes the prescription to a pharmacy, which then contacts the insurance carrier to determine if such prescription is allowed. A cycle often results where, if the prescription is not approved, the medical provider is contacted to recommend an alternative prescription, which is then sent to the pharmacy, which then again, must verify with the insurance carrier if it is permitted. Only in the instance where a prescription provided by a medical provider to the pharmacy, which is then subsequently approved by the insurance carrier, can a prescription be fulfilled. This interplay of prescribed courses of treatment (provided by a medical provider) which must be in alignment with what the pharmacy can provide and what the insurance carrier will cover, causes wasted time and resources, to say the least. Such inefficiencies are further exasperated when time-sensitive medical treatments are needed by the patient.

As such, there is thus a need for addressing these and/or other issues associated with the prior art.

### SUMMARY

A system, method, and non-transitory computer readable storage medium are provided for streamlining authorization for medical payment adjudication. In use, a treatment fulfillment request associated with a patient, and data associated with a medical model containing a ranking of at least two prescriptions associated with the treatment fulfillment request are received from a medical provider. Next, an adjudication status of at least one prescription of the at least two prescriptions is determined. When the adjudication status of one prescription of the at least two prescriptions indicates that the one prescription is allowed by the medical insurance provider, submit the one prescription for fulfillment to a pharmacy provider as a clean claim submission. When the adjudication status of each prescription of the at least two prescriptions indicates that each prescription of the at least two prescriptions is not allowed by the medical insurance provider, request additional data for the medical model from the medical provider.

### BRIEF DESCRIPTION OF THE DRAWINGS

FIG. 1 illustrates a method for streamlining authorization for medical payment adjudication, in accordance with one embodiment.

FIG. 2 illustrates system for interactions between medical related entities, in accordance with one embodiment.

FIG. 3 illustrates a method for determining an adjudication status, in accordance with one embodiment.

FIG. 4 illustrates a method for using a medical model to streamline authorization, in accordance with one embodiment.

FIG. 5 illustrates a method for using automated pre-adjudication workflows, in accordance with one embodiment.

FIG. 6 illustrates a method for payment or authorization approval applied at a fulfillment center, in accordance with one embodiment.

FIG. 7 illustrates a network architecture, in accordance with one possible embodiment.

FIG. 8 illustrates an exemplary system, in accordance with one embodiment.

### DETAILED DESCRIPTION

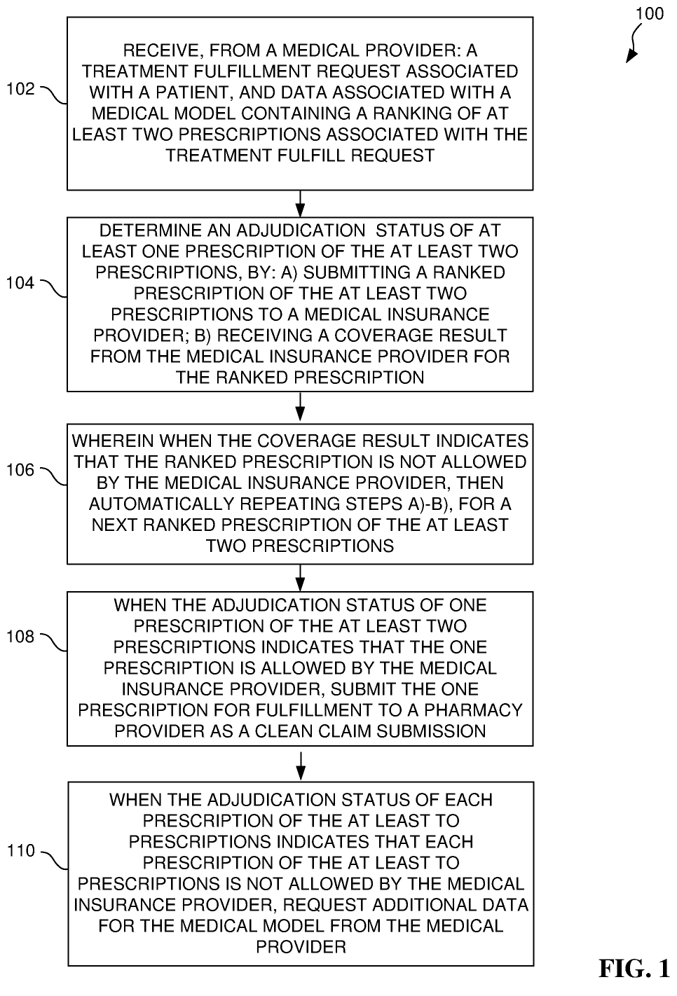

FIG. 1 illustrates a method 100 for streamlining authorization for medical payment adjudication, in accordance with one embodiment.

As shown, a treatment fulfillment request associated with a patient, and data associated with a medical model containing a ranking of at least two prescriptions associated with the treatment fulfillment request are received from a medical provider. See operation 102. In one embodiment, a treatment fulfillment request may include a prescription (or multiple prescriptions), a listing of ranked prescriptions, over-the-counter medicine, etc.

Additionally, data associated with a medical model may include preferred medication, medical allergy, preferred manner of communication, medical insurance associated with the patient, a pre-determined maximum price (by the patient, by the medical provider, etc.), personal attributes and preferences (associated with the patient or pharmacy, such as location, availability, price constraints, allergies, lifestyle, etc.), etc. In various embodiments, a medical model may include a therapeutic interchange, a medical and/or doctor decision tree, a medical and/or doctor algorithm, etc., where a medical treatment may be replaced, by a medical provider, with an alternative treatment. Further, the medical model may be automatic (alternative is presented automatically), semi-automatic (alternative may be presented contingent upon one or more conditions), and/or manual. In any case, the medical model may remain at the control of the medical provider. In other embodiments, the medical model may include one or more elements of a medical and/or decision tree, a medical and/or doctor algorithm, etc.

Additionally, an adjudication status of at least one prescription of the at least two prescriptions is determined by a) submitting a ranked prescription of the at least two prescriptions to a medical insurance provider; and b) receiving a coverage result from the medical insurance provider for the ranked prescription. See operation 104. Within the context of the present description, an adjudication status refers to a determination whether a prescribed product or service will be covered. For purposes of clarity, the adjudication status is a designation as to whether a prior authorization is required. For example, in one embodiment, a prescription may be submitted to determine if it is covered by the medical insurance provider. In response, the medical insurance can indicate: 1) it is a covered prescription; 2) it is not a covered prescription; or 3) it is a prescription that would require prior authorization. It is noted that the prior authorization process can conventionally go many different names, namely prior authorization, precertification, pre-authorization, prior approval, predetermination, etc. Within the context of the present description, prior authorization refers to any additional action that must be taken with a medical insurance provider associated with a prescription that is not either automatically indicated as being covered or not covered. As such, therefore, the adjudication status determines whether a prescription is allowed, not allowed, or would require any further action (such as prior authorization).

Conventionally, additional action (such as prior authorization) can be a long and often delayed process, which in some cases may cause the requested prescription to not be delivered to the patient within the timeframe that is needed (such as a planned surgery date, etc.). Embodiments herein therefore are intended to overcome the often slow, long and delayed process that are found in conventional prior authorization scenarios.

Further, when the coverage result indicates that the ranked prescription is not allowed by the medical insurance provider, then steps a)-b) are automatically repeated, for a next ranked prescription of the at least two prescriptions. See operation 106.

For example, in one embodiment, a list of ranked prescriptions may be provided by a medical provider for a patient. With respect to the first ranked prescription, it may be determined that the insurance company does not cover (in any way) that prescription. As such, the second ranked prescription may then be analyzed to determine coverage. It may be determined that the insurance company potentially cover (all or in part) the second ranked prescription, but that further action (submission via prior authorization) would be necessary. As such, the third ranked prescription may then be analyzed to determine coverage. It may be determined that the third ranked prescription would be covered (without having to go through prior authorization) by the medical insurance provider.

Thus, when the adjudication status of one prescription of the at least two prescriptions indicates that the one prescription is allowed by the medical insurance provider, submit the one prescription for fulfillment to a pharmacy provider as a clean claim submission. See operation 108. Alternatively, when the adjudication status of each prescription of the at least two prescriptions indicates that each prescription of the at least two prescriptions is not allowed by the medical insurance provider, request additional data from the medical provider. See operation 110.

For example, if all prescriptions of the at least prescriptions have been analyzed and none are covered by the medical insurance provider, additional data may include a second listing of prescriptions for which an adjudication status of the ranked prescriptions on the second listing can then be determined. For purposes of clarity, there is no limit to the data associated with the medical model that is received. Thus, the medical provider may provide any number of preferred prescriptions and acceptable alternatives. Should at least one of the prescriptions or acceptable alternatives on the first listing not be approved, the medical provider may then have the opportunity to provide additional alternatives, or to contact the patient to determine if any modification to the data associated with the medical model is needed (e.g. increase a price ceiling amount, remove location limitation, extend time restriction, etc.). As such, the secondary listing may include new prescriptions and/or alternatives, and/or may include updated factors (such as pricing, location, timing considerations, etc.) which may influence the adjudication status determination.

In one embodiment, determining the adjudication status may include submitting the at least two prescriptions to an automated pre-adjudication workflow. Additionally, when a covered prescription of the at least two prescriptions is allowed by the medical insurance provider, a pre-approved signature from the medical provider may be applied to the covered prescription prior to the covered prescription being submitted to the pharmacy provider. For example, if a non first ranked prescription is allowed (and ready to be submitted to the pharmacy provider), a signature from the medical provider (doctor) may be applied to the covered prescription. Of course, it is to be appreciated that applying the medical provider's signature to any prescription remains under the control and direction of the medical provider. As such, pre-approval from the medical provider (to sign on behalf of the medical provider for the first allowed prescription) may be received as a prerequisite prior to processing of any of the prescriptions provided in the treatment fulfillment request.

Further, the ranking of the at least two prescriptions may be based on input from the medical provider, and/or on likelihood of success with the medical insurance provider. For example, the input of the medical provider may include a personal preference, a judgment based on criteria determined with the medical provider and the patient, past history of success using such prescriptions, etc. Further, the ranking may be based at least in part on a likelihood of success with the medical insurance provider (e.g. historically the medical insurance provider may or may not cover such prescription). That being said, it is to be appreciated that coverage of a prescription is based not only on the medical insurance provider generally but also on a coverage plan of the patient of the medical insurance provider. In this manner, two individuals with the same coverage plan through a medical provider may each have a different outcome (allowed, not allowed, prior authorization required) despite having the same coverage plan. As such, the likelihood of success may take into consideration data metrics across multiple patients (e.g. all patients associated with a particular medical insurance provider had a particular prescription deemed not covered, etc.). Such data metrics may also take into account the lifecycle of a drug (e.g. newly released, coupons available, available in mainstream pharmacies, generic version available, etc.).

In one embodiment, the data associated with a medical model may include information associated with the patient including at least one of: employment, education, income, race, ethnicity, prior medical condition, family medical history, insurance coverage, age, gender, nationality, ancestry, or religion. As such, demographic information associated with the patient may be provided and/or inputted. In one embodiment, the demographic information may include any information needed for the submission of compliance forms to the medical insurance provider associated with the patient. As such, the data gathered may be a function of the medical insurance provider (in that each medical insurance provider may have certain predetermined criteria that must be included with a coverage submission request).

Additionally, in one embodiment, payment or authorization approval by the patient in relation to the treatment fulfillment request may be received from the medical provider. For example, a patient may visit a medical provider, and the medical provider may recommend a particular prescription (or course of treatment). Payment for the particular prescription (or allowance for a price up to a certain amount) may be received by the medical provider. This payment or authorization approval may include a pre-authorization request (with a credit card company) where a temporary hold on expected funds (needed to cover the prescription) may be provided. Once the prescription is deemed covered by the medical insurance provider, and a clean claim submission is sent to the pharmacy provider, the pharmacy provider can act as the merchant for the purposes of completing and finalizing the pre-authorization request (assuming that the final price for the allowed prescription is the same as or below the price limit authorized by the patient). In this manner, a clean claim submission to a pharmacy may include all logistical components (insurance coverage, demographic information, payment, etc.) completed, which in turn may significantly reduce the amount of time a pharmacy provider has to spend in processing and fulfilling a prescription.

As such, when a covered prescription of the at least two prescriptions is allowed by the medical insurance provider, the payment or authorization approval may be sent to the pharmacy provider. Further, the payment or authorization approval may include a token. In this manner, a tokenized transaction relating to the payment may occur, which may be initially generated by the medical provider and eventually finalized (transacted) by the pharmacy provider. Within the context of the present description, a tokenized transaction relating to the payment may include receiving payment information (e.g. credit card information, debit card information, cash provided, etc.) and replacing the content of the payment information with random strings of numbers and letters. In this manner, the token may preserve the security of the payment or authorization approval until the pharmacy provider is ready to finalize and fulfill the allowed prescription.

In one embodiment, the pharmacy provider may include an online provider. For example, the pharmacy provider may receive prescription requests and fulfill those prescription requests by mailing out the fulfilled prescription. In another embodiment, the pharmacy provider may be an on-site provider. For example, the pharmacy provider may receive prescription request and fulfill those prescription requests by providing a pick-up location site for the fulfilled prescription, where the patient can physically go to pick up the fulfilled prescription.

Further, in one embodiment, the pharmacy provider may be selected based on at least one of: a pre-indicated preference from the patient, a geographic distance between the pharmacy provider and the patient, a coupon available at the pharmacy provider, or open hours of the pharmacy provider. In this manner, the pharmacy provider may be selected based on pricing considerations, location considerations, personal preference considerations, all of which may be associated with the patient and/or the medical provider. In another embodiment, the criteria provided herein may be included as data associated with the medical model (and used in the determination of the adjudication status). As such, criteria used to select a pharmacy provider may be used within the determination of the adjudication status, and/or may be used later in the process (once a clean claim submission is ready).

In one embodiment, the coverage result may include a computation of payment from at least two of: the medical insurance provider, patient payment, or available coupon. Further, the computation may include a ceiling limit amount defined by the patient payment. For example, in one scenario, a prescription may cost $125 (in acquisition costs). The medical insurance provider may provide a capped amount (such as $125) to cover the cost of the prescription, and may dictate that the patient pay a certain amount (such as $50 in co-pay amount), and the remaining amount ($75) covered by the medical insurance provider. A coupon may be available for the prescription ($40) such that the patient payment amount may thereby decrease (to $10). Thus, a combination of patient amount ($10), plus the coupon amount ($40), plus the medical insurance provider ($75) may in aggregate sum to the total acquisition cost as dictated by the desired prescription. In some instances, the patient may have a capped amount that they direct that they can pay (such as $5). Based on such indicated capped amount, the computation of payment will determine if the acquisition cost can be satisfied by the summation of the patient's contribution, any coupons available, and the allowed covered amount by the medical insurance provider.

If the computation of payment is not satisfied by such constraints, then the prescription may be labeled as not a clean claim submission (and the next ranked prescription may then be evaluated). If the patient were to modify the capped amount (such as up to $20), the computation may be recomputed to determine if the raised ceiling limit amount now allows a prescription to be fully covered (i.e. all acquisition costs are satisfied as dictated by the breakdown in payment from the medical insurance provider).

In one embodiment, the clean claim submission may include at least two of a pre-approval designation for a covered prescription with the medical insurance provider, a verification of demographic information associated with the patient, an approval from the patient for the covered prescription, and/or a signature from the medical provider for the covered prescription. In this manner, the adjudication negotiation process that the pharmacy provider may otherwise have to engage in may be simplified (and time reduced) in that the approvals, compliance, coverage, and verification associated with any or all of the medical provider, the patient, and/or the medical insurance provider may be done in advance before sending the clean claim submission to the pharmacy provider.

Further, in one embodiment, determining an adjudication status of the at least one of the at least two prescriptions may include a time sensitivity for the at least one of the at least two prescriptions. For example, if the first ranked prescription cannot be obtained and delivered to the patient within a predetermined timeframe (such as within 6 hours, within 24 hours, within 48 hours, etc.), then it may be designated as not covered by the medical insurance provider. Additionally, if a medical event (such as a surgery) requires a prescription, the timing of the medical event may be used as hard date to determine what prescriptions can be obtained that are compliant with the treatment fulfillment request (including necessary timing).

Still yet, in one embodiment, the clean claim submission may include documentation for legal compliance for the pharmacy provider. Additionally, receiving the coverage result from the medical insurance provider for the first ranked prescription may, in one embodiment, not include submitting the first ranked prescription to a prior authorization process associated with the medical insurance provider. In this manner, the data associated with the medical model may avoid having to submit the desired prescription to a prior authorization process associated with the medical insurance provider. Further, the coverage result may include a pre prior authorization indication. For example, the pre prior authorization indication may include a designation of whether the desired prescription would be covered for the patient by the medical insurance provider, would not be covered for the patient by the medical insurance provider, and/or would require further analysis (via a prior authorization process or step edit process) for the patient by the medical insurance provider.

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described.

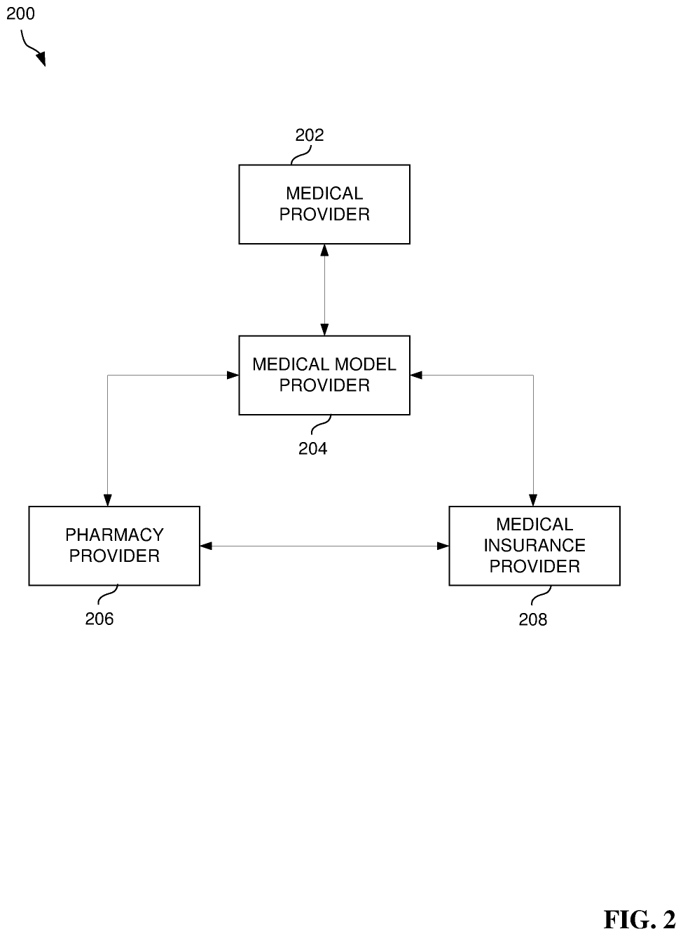

FIG. 2 illustrates a system 200 for interactions between medical related entities, in accordance with one embodiment. As an option, the system 200 may be implemented in the context of any one or more of the embodiments set forth in any previous and/or subsequent figure(s) and/or description thereof. Of course, however, the system 200 may be implemented in the context of any desired environment. Further, the aforementioned definitions may equally apply to the description below.

As shown, a medical model provider 204 is in communication with a medical provider 202, a pharmacy provider 206, and a medical insurance provider 208. Data may be exchanged between the medical provider 202 and the medical model provider 204. For example, the data sent from the medical provider 202 may include information from the medical provider 202 such as demographic information, a listing of recommended prescriptions, constraints (pricing, location, lifestyle, etc.) associated with the patient, etc. Data sent from the medical model provider 204 may include a request for additional information, a request for a secondary listing of prescriptions, a result of an adjudication status of previously submitted prescriptions, etc.

Additionally, data may be exchanged between the pharmacy provider and the medical model provider 204. For example, when a prescription is determined to be allowed, it may be submitted to the pharmacy provider as a clean claim submission. Such submission would include a request for fulfillment, information needed (for the fulfillment) associated with the patient, insurance coverage information (including any information and/or documentation needed to comply with insurance compliance forms and/or obligations, etc.). In response, the pharmacy provider 206 may indicate back to the medical model provider 204 that the request was received and/or fulfilled. The pharmacy provider 206 may additionally indicate if any issue surfaced in the fulfillment of a covered prescription, such as the prescription no longer being available, an alternative to requested prescription, a change in a condition of the request (e.g. a coupon amount changed, the insurance coverage suddenly no longer deems the prescription as allowed, etc.). As such, information may be sent from the pharmacy provider 206 to the medical model provider 204. In some instances, based on the information sent from the pharmacy provider 206 to the medical model provider 204, the medical model provider 204 may need to take additional action (e.g. find an alternative prescription that is covered, etc.).

Further, data may be exchange between the medical insurance provider 208 and the medical model provider 204. For example, the medical model provider 204, based on a listing of ranked prescriptions received from the medical provider 202, may reach out to the medical insurance provider to determine if any of the prescriptions (of the listing of ranked prescriptions) is allowed by the medical insurance provider 208. In this manner, the medical insurance provider 208 may provide feedback to the medical model provider 204 as to whether a prescription would be allowed, not allowed, and/or require further action (prior authorization, etc.).

As displayed, the pharmacy provider 206 may also exchange data (and/or otherwise be in contact with) the medical insurance provider 208. For example, as part of the fulfillment of the prescription, the pharmacy provider 206 may fill out and/or complete required forms (from the medical insurance provider 208) and submit such forms and/or other compliant documentation to the medical insurance provider for processing of the insurance claim. The medical insurance provider 208 in turn may reach out to the pharmacy provider 206 for verification (of the claim), for clarification, and/or to provide feedback in relation to the submission.

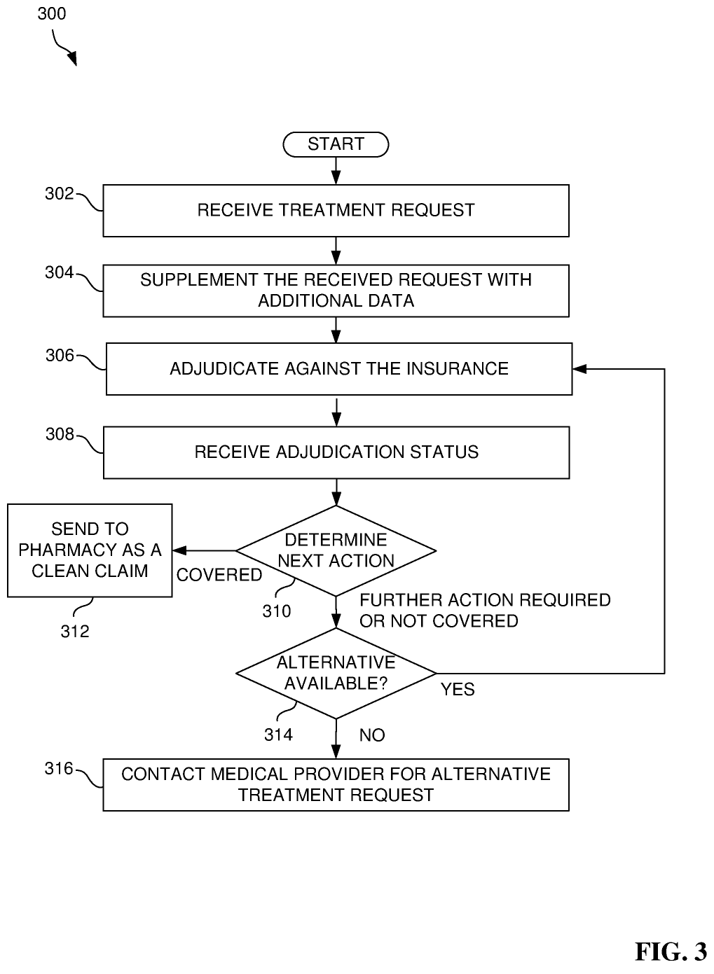

FIG. 3 illustrates a method 300 for determining an adjudication status, in accordance with one embodiment. As an option, the method 300 may be implemented in the context of any one or more of the embodiments set forth in any previous and/or subsequent figure(s) and/or description thereof. Of course, however, the method 300 may be implemented in the context of any desired environment. Further, the aforementioned definitions may equally apply to the description below.

As shown, the method 300 starts with receiving a treatment request. See operation 302. The treatment request may be received electronically (including in digital format) and/or any other communication transmission (e.g. facsimile, paper request, etc.). In one embodiment, the treatment request of operation 302 may be sent from the medical provider 202 to the medical model provider 204.

Next, in operation 304, the received treatment request may be supplemented with additional data. For example, the received treatment request may be supplemented with demographic information associated with the patient, specific information required by the pharmacy provider (e.g. to ensure compliance, etc.) and/or the medical insurance provider associated with the patient, etc.

Next, the treatment request may be adjudicated against the insurance. See operation 306. In response, an adjudication status may be received. See operation 308. The adjudication process may include determining whether the requested prescription is allowed, not allowed, and/or requires further action (e.g. prior authorization, etc.), and providing a response (an adjudication status) to that effect.

Based on the adjudication status, it is determined what the next action is. See decision 310. For example, if a prescription is covered, the prescription is sent to a pharmacy provider as a clean claim submission. See operation 312. See also operation 108 for additional context.

If a prescription is not covered, it is determined if an alternative is available. See decision 314. In one embodiment, if a prescription is determined not to be covered, rather than go back to the medical provider, the next alternative prescription (assuming a listing of prescriptions were provided with the treatment request per operation 302) may then be analyzed (returns to operation 306). If no other alternatives exist, then the method 300 continues on to operation 316 where the medical provider may be contacted to provide an alternative (or a listing of ranked alternatives) that would work for the patient.

In this manner, a listing of prescriptions from a medical provider may be analyzed to determine if one of the prescriptions is covered by the medical insurance provider. If none of the prescriptions are allowed, then the medical provider can be contacted to determine if an alternative treatment request is needed. As discussed hereinabove, a modification (e.g. price ceiling, etc.) may be modified which in turn may alter the adjudication process. Further, based on this analysis, information can be provided back to the patient to determine if prior authorization is the best option forward. For example, based on the adjudication status (per operation 308) for all prescriptions provided, it may be determined that no other alternative exists. Thus, in that instance, the patient may decide to go through the time-intensive process of prior authorization with the medical insurance provider. However, such decision may be made after all other options and alternatives have been fully explored.

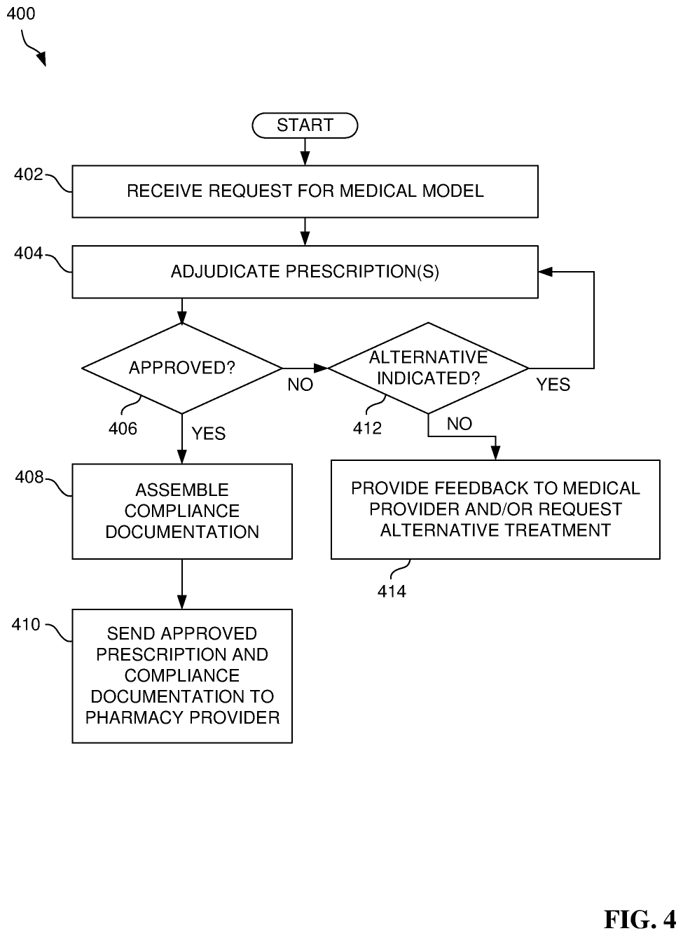

FIG. 4 illustrates a method 400 for using a medical model to streamline authorization, in accordance with one embodiment. As an option, the method 400 may be implemented in the context of any one or more of the embodiments set forth in any previous and/or subsequent figure(s) and/or description thereof. Of course, however, the method 400 may be implemented in the context of any desired environment. Further, the aforementioned definitions may equally apply to the description below.

As shown, the method 400 begins with receiving a request for medical model. See operation 402. Next, the prescription (indicated in the request) may be adjudicated. See operation 404. It is determined whether prescription is approved by the medical insurance provider. See decision 406. If yes, compliance documentation (such as required forms and/or information needed for medical insurance provider compliance) may be assembled. See operation 408. Next, the approved prescription and compliance documentation are sent to the pharmacy provider. See operation 410.

If the prescription is not approved, it is determined if an alternative is indicated. See decision 412. If yes, the method 400 returns back to operation 404 to adjudicate the next indicated prescription. If no, then feedback is provided back to the medical provider and/or a request for alternative treatment. See operation 414.

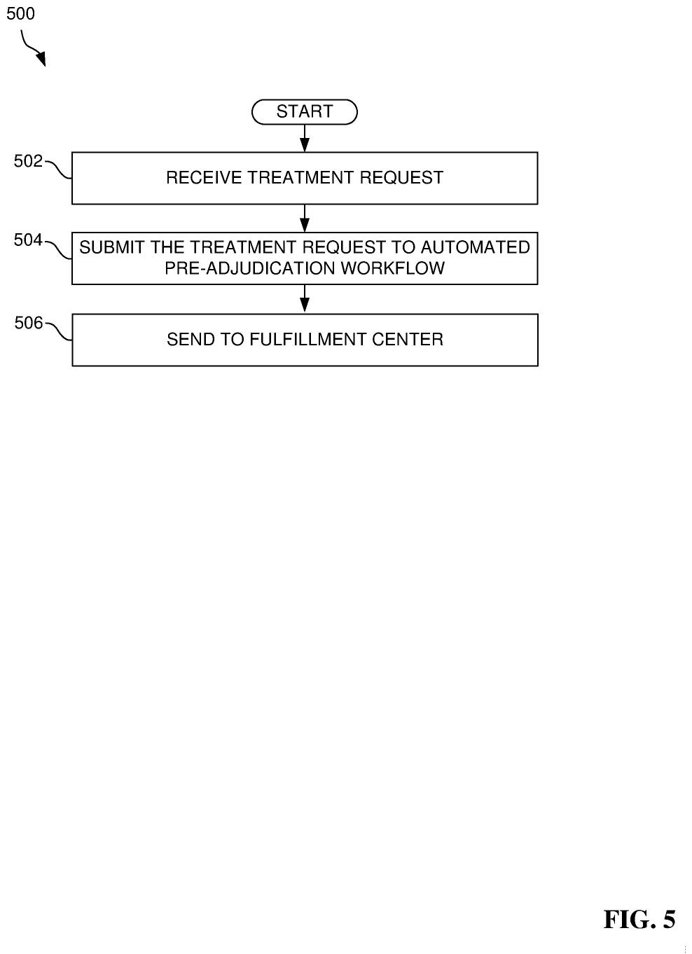

FIG. 5 illustrates a method 500 for using automated pre-adjudication workflows, in accordance with one embodiment. As an option, the method 500 may be implemented in the context of any one or more of the embodiments set forth in any previous and/or subsequent figure(s) and/or description thereof. Of course, however, the method 500 may be implemented in the context of any desired environment. Further, the aforementioned definitions may equally apply to the description below.

As shown, a treatment request is received. See operation 502. Next, the treatment request may be submitted to an automated pre-adjudication workflow. See operation 504. Within the context of the present description a pre-adjudication workflow refers to any process to determine whether a prescription (or course of treatment) is approved by the medical insurance provider without having to submit such prescription (or course of treatment) to a prior authorization review process.

The pre-adjudication workflow may include several automated type aspects. For example, in one embodiment, it may be determined that the treatment request involves an over-the-counter medicine where it is not needed to get a medical insurance provider involved. Based on such, the medical model provider may determine an appropriate fulfillment center to fill the over-the-counter medicine. In other embodiments, a treatment request may include multiple over-the-counter medicines and prescriptions, and the medical model provider may analyze the treatment request to optimize fulfillment. For example, it may be determined that the over-the-counter medicines can be sent out immediately (or be made immediately available for pickup). In other embodiments, it may be determined that the patient rated convenience of delivery high, so all of the over-the-counter medicines and the prescriptions may be sent to a single fulfillment center (e.g. pharmacy provider) for delivery and/or pickup. In this manner, therefore, the medical model provider may optimize fulfillment of all needed items dictated in the treatment request from the medical provider.

Based on the pre-adjudication workflow, the treatment request is sent to a fulfillment center. See operation 506. In one embodiment, the fulfillment center may include a pharmacy provider. In other embodiments, if the treatment request included non prescription treatment, the fulfillment center may include a provider for over-the-counter medicines.

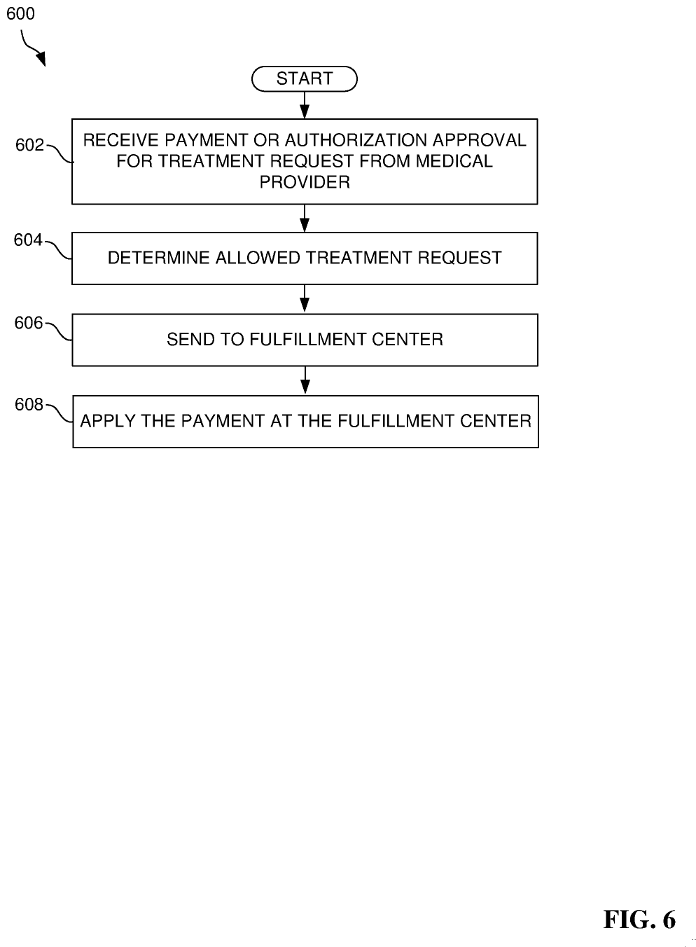

FIG. 6 illustrates a method 600 for payment or authorization approval applied at a fulfillment center, in accordance with one embodiment. As an option, the method 600 may be implemented in the context of any one or more of the embodiments set forth in any previous and/or subsequent figure(s) and/or description thereof. Of course, however, the method 600 may be implemented in the context of any desired environment. Further, the aforementioned definitions may equally apply to the description below.

As shown, a payment or authorization approval for treatment request from a medical provider may be received. See operation 602. As described herein, the payment or authorization approval may include a token and/or a pre-authorization (or authorization hold) indication.

Next, it is determined whether the treatment request is allowed. See operation 604. For example, allowance of a treatment request may occur consistent with operation 104 described herein. The treatment request may then be sent to a fulfillment center. See operation 606.

At the fulfillment center, the payment may then be applied. See operation 608. In this manner, payment may be initially received by the medical provider (at the time the recommended course of treatment is given), and the payment may be affected at the time of fulfillment by the fulfillment center.

In various embodiments, the medical model provider may determine that the treatment request from the medical provider does not require interaction with a medical insurance provider. In such instance, the medical model provider may send the treatment request to a pharmacy provider, may fulfill the treatment request, and/or may send to a third-party fulfillment center. As such, in the case of a treatment request that includes a non prescription treatment, fulfillment of the non prescription treatment may occur via any available fulfillment provider without having to interact with a medical insurance provider. Further, the medical model provider may function, in some instances, as a pharmacy provider (for other the counter type medicines).

In other embodiments, the medical model provider may include a medical model system that includes pre-approved logic based on instructions from the medical provider. For example, the medical provider may indicate a preferred prescription, which if not covered by the medical insurance provider, a first alternative may be used, which if not covered by the medical insurance provider, a second alternative may be used, and so on and so forth. In this manner, therefore the medical model may be used to determine, based on a ranking provided by a medical provider, a first match between a ranked prescription and coverage with the medical insurance provider. If all alternatives have been explored with the medical model with none resulting in a covered prescription by the medical insurance provider, then a listing of further alternatives can be provided by the medical provider, a modification to the adjudication constraints (pricing, etc.) can be provided, and/or the medical provider in communication with the patient may decide to pursue prior authorization as needed.

In various embodiments, the adjudication process may include determining what party is paying for what. The adjudication process may include running a claim to determine allocation of costs among the medical insurance provider, the patient, and any available coupons that can be applied.

In the instance where a prescription is covered by a medical insurance provider, it can be labeled as a clean claim submission and submitted to a fulfillment provider. In one embodiment, the clean claim submission may indicate that it is fully covered (paid fully by the medical insurance provider, or paid by a combination of any of the medical insurance provider, the patient, and/or coupons available). In some embodiments, a medical provider may include a treatment (e.g. prescription, medicine, etc.) as part of an inclusive procedure (e.g. treatment is included in part of a surgery, etc.). In such an instance, the treatment part of the inclusive procedure may be treated as a cash type payment (as there is no need to adjudicate with the medical insurance provider).

In one embodiment, the medical model provider may facilitate payment, correspond with the patient, obtain additional information (demographic) associated with the patient, receive authorization (to proceed forward with an indicated course of treatment from the medical provider), etc. Such information may be forwarded on to a partner pharmacy provider. In this manner, the pharmacy provider may not need to correspond with the patient (in contrast to conventional methods) as all details (relating to medical provider coverage, demographic information, etc.) may be previously resolved by the medical model provider. As such, in combination with the medical model provider, the pharmacy provider can function as a fulfillment provider (without having to determine coverage status, etc.). It is noted that even in combination with the medical model provider, the pharmacy provider may still be responsible to comply with compliance requirements (legal, regulatory, etc.). Nonetheless, such compliance requirements can be alleviated significantly (with time savings) by partnering with the medical model provider.

In the instance where a prescription requires further action (such as prior authorization), the patient can decide whether to go through the prior authorization process and/or to pursue an alternative. For example, a generic version of the branded prescription that was adjudicated may be selected by the patient as an alternative. In some embodiments, typical questions that are used (in a step edit, as part of prior authorization, etc.) may include: “what is the diagnosis”; “what have you tried and failed”; “are you qualified to take this medication?” These questions, in one embodiment, may be pursued prior to the prior authorization process by the medical model, such that all alternatives may be pursued and known without having to engage in a step edit process with the medical insurance provider.

In various embodiments, the medical model provider may manage, at least in part, compliance forms for medical insurance providers. For example, if a form is needed for submission by the pharmacy provider to the medical insurance provider, the medical model provider may assist in either completing such form, or providing any necessary information needed for the form to the pharmacy provider. In other instances, if a prior authorization is desired to be pursued (after alternatives have already been exhausted), the medical model may assist with finding the correct form for the medical insurance provider and filling it out as needed to satisfy any compliance requirements (legal, regulatory, internal, insurance, etc.). In this manner, the medical model provider can facilitate compliance requirements for, in particular, the pharmacy provider and/or for the patient (if going through the prior authorization process).

Additionally, the compliance may include state specific compliance regulations as well. For example, a state may require written communication from a medical provider. The medical model provider may provide documentation as needed to satisfy state specific compliance regulations. In other instances, a medical insurance provider may require an original prescription from the medical provider. In such instances, the medical provider may provide authorization (on a grouping of prescriptions) such that when a first prescription is found to be covered by the medical insurance provider a signature may be applied (per the authorization given with the grouping of prescriptions) to the covered prescription prior to sending the submission request to the pharmacy provider. In this manner, the burden imposed by specific compliance regulations of the medical insurance provider may also be alleviated by the medical model provider. As such, written proof, documentation, original signatures, and/or any other regulatory compliance requirements may be provided by the medical model provider based on their communication with the medical provider, the medical insurance provider, and the pharmacy provider.

In the instance where a prescription is not covered (and no alternative is covered), the medical provider can review to determine if another alternative can be submitted for adjudication. Additionally, the medical provider can determine if any constraints (on pricing, location, lifestyle) associated with the patient and/or the pharmacy provider can be modified, and based on such modifications, the same set of prescriptions can be submitted for adjudication once again.

In various embodiments, a time sensitivity may be associated with a prescription. For example, a patient may have need for a prescription based on a surgery date (hard date with no flexibility) or a medical event (such as glaucoma where one could lose their eyesight). In such embodiments, in particular, methods disclosed here using the medical model may significantly reduce the time needed to obtain an approval for a prescription that will be based on the treatment fulfillment request provided by the medical provider. Of course, it is to be appreciated that even non urgent medical events may benefit from using the medical model disclosed herein, as most people with a medical event still have a subjective and personal time sensitivity (i.e. they wish to resolve the medical issue sooner rather than later).

Still yet, in one embodiment, thresholds may be used in combination with the medical model. For example, a drug A may be a preferred first choice by the medical provider, but it may not be covered by the medical insurance provider. A coupon may be available for drug A that will reduce the price by 50%. Such feedback may be provided back to the medical provider and the patient such that the patient may decide to proceed forward with drug A at 50% cost without having to go through an insurance company (in which case it can be processed as a clean claim submission to the pharmacy provider). As another outcome, the patient may decide that 50% cost is still too much of a burden, and may work with the medical provider to find another alternative that 1) can be covered by the medical insurance provider; and/or 2) has a coupon available where the overall price for the patient is lower. As such, thresholds on pricing may be handled on a case-by-case basis with the medical provider and the patient. In other embodiments, the patient may provide thresholds to the medical provider (such as a price up to $50 can be paid by the patient, etc.) which in turn can be used automatically in the adjudication process that the medical model goes through.

In various embodiments, logic and rules may be used to determine the appropriate pharmacy provider. For example, a patient may indicate a preferred pharmacy. However, a non preferred pharmacy may provide a lower overall cost to the patient, and lower cost may be of higher priority than use of a preferred pharmacy. As such, having a priority listing of what the patient desires may allow the medical model to send the prescription out to the pharmacy provider that provides the best fit based on the rules provided. In some instances, thresholds may be used to trigger the need to reach out to the patient. For example, a patient may indicate that the highest priority is to use a preferred pharmacy. However, another non preferred pharmacy may offer the same prescription at a 50% cost reduction. The threshold of (at least a 40% cost reduction) may have been inputted by the patient such that when the threshold is satisfied, the medical model (on a case-by-case basis, or automatically, as instructed by the patient) may reach out to the patient to determine whether the patient wishes to pursue an alternative pharmacy provider based on a threshold having been surpassed.

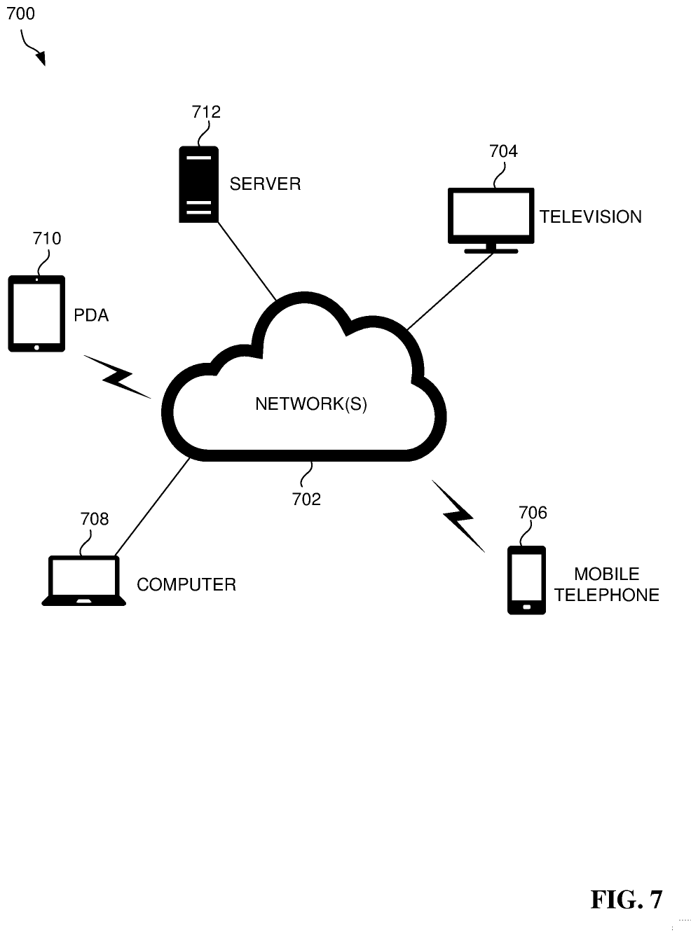

FIG. 7 illustrates a network architecture 700, in accordance with one possible embodiment. As shown, at least one network 702 is provided. In the context of the present network architecture 700, the network 702 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 702 may be provided.

Coupled to the network 702 is a plurality of devices. For example, a server computer 712 and an end user computer 708 may be coupled to the network 702 for communication purposes. Such end user computer 708 may include a desktop computer, lap-top computer, and/or any other type of logic. Still yet, various other devices may be coupled to the network 702 including a personal digital assistant (PDA) device 710, a mobile phone device 706, a television 704, etc.

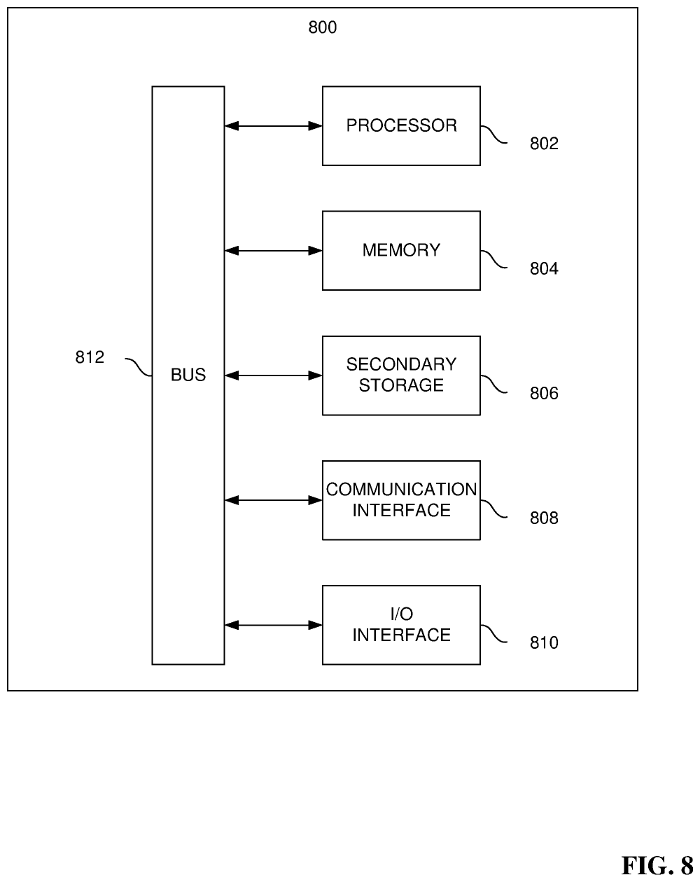

FIG. 8 illustrates an exemplary system 800, in accordance with one embodiment. As an option, the system 800 may be implemented in the context of any of the devices of the network architecture 700 of FIG. 7. Of course, the system 800 may be implemented in any desired environment.

As shown, a system 800 is provided including at least one central processor 802 which is connected to a communication bus 812. The system 800 also includes main memory 804 [e.g. random access memory (RAM), etc.]. The system 800 also includes a graphics processor 808 and a display 810.

The system 800 may also include a secondary storage 806. The secondary storage 806 includes, for example, a hard disk drive and/or a removable storage drive, representing a floppy disk drive, a magnetic tape drive, a compact disk drive, etc. The removable storage drive reads from and/or writes to a removable storage unit in a well known manner.

Computer programs, or computer control logic algorithms, may be stored in the main memory 804, the secondary storage 806, and/or any other memory, for that matter. Such computer programs, when executed, enable the system 800 to perform various functions (as set forth above, for example). Memory 804, storage 806 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

In various embodiments, the medical model may be used and driven by a variety of entities. For example, as explained herein, the medical model may be invoked in response to a medical insurance provider (for purposes of determining if a prescription is covered by a specific medical insurance plan associated with a patient). In other embodiments, the medical model may be used in response to a cost-savings need where, for example, if a claim is covered by insurance but the cost is still prohibitive for a patient, it may be advised to move to a treatment that is more affordable for the patient. Thus, cost savings on behalf of the patient may also trigger use of the medical model.

In another embodiment, compliance consideration(s) may drive use of the medical model. For example, some medications require dosing once daily, 4 times per day, etc. The more frequent the dosing the less likely a patient may remain compliant. Thus, the medical model may be used to ensure greatest likelihood of compliance with a patient. As indicated herein, however, the medical model may be patient driven (based on patient input and/or metrics) and/or may include patient interaction. The patient may participate therefore in their own healthcare decisions. In this manner, the patient remains the ultimate consumer in that they can dictate what is going to happen. Price, allergies, dosing and other factors may be considered by the patient before a decision (directed by the patient) may be made. As such, the use of the medical model may allow for a more true collaboration between patient, medical provider (doctor), medical insurance provider, and/or pharmacy partner in the final outcome of such interactions and collaboration may be patient-driven, patient-directed, and ensure the patient's best interest.

As used here, a “computer-readable medium” includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A non-transitory computer readable storage medium storing one or more programs, the one or more programs comprising instructions which, when executed by an apparatus, cause the apparatus to:
receive, from a medical provider:
a treatment fulfillment request associated with a patient, and
data associated with a medical model containing a ranking of at least two prescriptions associated with the treatment fulfillment request;
determine an adjudication status of at least one prescription of the at least two prescriptions, by:
a) submitting a ranked prescription of the at least two prescriptions to a medical insurance provider;
b) receiving a coverage result from the medical insurance provider for the ranked prescription, wherein when the coverage result indicates that the ranked prescription is not allowed by the medical insurance provider, then automatically repeating steps a)-b), for a next ranked prescription of the at least two prescriptions;
when the adjudication status of one prescription of the at least two prescriptions indicates that the one prescription is allowed by the medical insurance provider, submit the one prescription for fulfillment to a pharmacy provider as a clean claim submission; and
when the adjudication status of each prescription of the at least two prescriptions indicates that each prescription of the at least two prescriptions is not allowed by the medical insurance provider, request additional data for the medical model from the medical provider.

2. The non-transitory computer readable storage medium of claim 1, wherein determining the adjudication status includes submitting the at least one prescription of the at least two prescriptions to an automated pre-adjudication workflow.

3. The non-transitory computer readable storage medium of claim 2, wherein when the one prescription of the at least two prescriptions is allowed by the medical insurance provider, apply a pre-approved signature from the medical provider to the one prescription prior to the one prescription being submitted to the pharmacy provider.

4. The non-transitory computer readable storage medium of claim 1, wherein the ranking is based on input from the medical provider.

5. The non-transitory computer readable storage medium of claim 1, wherein the ranking is based on likelihood of success with the medical insurance provider.

6. The non-transitory computer readable storage medium of claim 1, wherein the data includes information associated with the patient including at least one of: employment, education, income, race, ethnicity, prior medical condition, family medical history, insurance coverage, age, gender, nationality, ancestry, or religion.

7. The non-transitory computer readable storage medium of claim 1, wherein the one or more programs comprises instructions which, when executed by the apparatus, cause the apparatus to:
receive, from the medical provider, payment or authorization approval provided by the patient in relation to the treatment fulfillment request; and
when the adjudication status of the one prescription of the at least two prescriptions indicates that the one prescription is allowed by the medical insurance provider, send the payment or authorization approval to the pharmacy provider.

8. The non-transitory computer readable storage medium of claim 7, wherein the payment or authorization approval includes a token.

9. The non-transitory computer readable storage medium of claim 1, wherein the pharmacy provider is an online provider.

10. The non-transitory computer readable storage medium of claim 1, wherein the pharmacy provider is an on-site provider.

11. The non-transitory computer readable storage medium of claim 1, wherein the pharmacy provider is selected based on at least one of: a pre-indicated preference from the patient, a geographic distance between the pharmacy provider and the patient, a coupon available at the pharmacy provider, or open hours of the pharmacy provider.

12. The non-transitory computer readable storage medium of claim 1, wherein the coverage result includes a computation of payment from at least two of: the medical insurance provider, a patient payment, or an available coupon.

13. The non-transitory computer readable storage medium of claim 12, wherein the computation includes a ceiling limit amount defined by the patient payment.

14. The non-transitory computer readable storage medium of claim 1, wherein the clean claim submission includes at least two of:
a pre-approval designation for the one prescription with the medical insurance provider;
a verification of demographic information associated with the patient;
an approval from the patient for the one prescription; or
a signature from the medical provider for the one prescription.

15. The non-transitory computer readable storage medium of claim 1, wherein determining an adjudication status of the at least one of the at least two prescriptions includes a time sensitivity for the at least one prescription of the at least two prescriptions.

16. The non-transitory computer readable storage medium of claim 1, wherein the clean claim submission includes documentation for legal compliance for the pharmacy provider.

17. The non-transitory computer readable storage medium of claim 1, wherein receiving the coverage result from the medical insurance provider for the ranked prescription does not include submitting the ranked prescription to a prior authorization process associated with the medical insurance provider.

18. The non-transitory computer readable storage medium of claim 1, wherein the coverage result is a pre prior authorization indication.

19. A computer-implemented method, comprising:
receiving, from a medical provider:
a treatment fulfillment request associated with a patient, and
data associated with a medical model containing a ranking of at least two prescriptions associated with the treatment fulfillment request;
determining an adjudication status of at least one prescription of the at least two prescriptions, by:
a) submitting a ranked prescription of the at least two prescriptions to a medical insurance provider;
b) receiving a coverage result from the medical insurance provider for the ranked prescription, wherein when the coverage result indicates that the ranked prescription is not allowed by the medical insurance provider, then automatically repeating steps a)-b), for a next ranked prescription of the at least two prescriptions;
when the adjudication status of one prescription of the at least two prescriptions indicates that the one prescription is allowed by the medical insurance provider, submit the one prescription for fulfillment to a pharmacy provider as a clean claim submission; and
when the adjudication status of each prescription of the at least two prescriptions indicates that each prescription of the at least two prescriptions is not allowed by the medical insurance provider, request additional data for the medical model from the medical provider.

20. A device, comprising:
a non-transitory memory storing instructions; and
one or more processors in communication with the non-transitory memory, wherein the one or more processors execute the instructions to:
receive, from a medical provider:
a treatment fulfillment request associated with a patient, and
data associated with a medical model containing a ranking of at least two prescriptions associated with the treatment fulfillment request;
determine an adjudication status of at least one prescription of the at least two prescriptions, by:
a) submitting a ranked prescription of the at least two prescriptions to a medical insurance provider;
b) receiving a coverage result from the medical insurance provider for the ranked prescription, wherein when the coverage result indicates that the ranked prescription is not allowed by the medical insurance provider, then automatically repeating steps a)-b), for a next ranked prescription of the at least two prescriptions;
when the adjudication status of one prescription of the at least two prescriptions indicates that the one prescription is allowed by the medical insurance provider, submit the one prescription for fulfillment to a pharmacy provider as a clean claim submission; and
when the adjudication status of each prescription of the at least two prescriptions indicates that each prescription of the at least two prescriptions is not allowed by the medical insurance provider, request additional data for the medical model from the medical provider.
